(19) United States
(12) Patent Application Publication
(10) Pub. No.: US 2026/0013807 A1
(43) Pub. Date: Jan. 15, 2026

(54) Patient Call Ball, Magnetic Resonance Apparatus Having a Patient Call Ball, and Method for Using a Patient Call Ball for a Magnetic Resonance Examination

(71) Applicant: Siemens Healthineers AG, Forchheim (DE)

(72) Inventor: Stephan Biber, Erlangen (DE)

(73) Assignee: Siemens Healthineers AG, Forchheim (DE)

(21) Appl. No.: 19/265,080

(22) Filed: Jul. 10, 2025

(30) Foreign Application Priority Data
Jul. 11, 2024 (EP) 24188010.3

(51) Int. Cl. A61B 5/00; A61B 5/055; G01R 33/28; H04W 4/80

(52) U.S. Cl. CPC A61B 5/7465; A61B 5/055; G01R 33/283; G01R 33/288; H04W 4/80

(57) ABSTRACT

A patient call ball may be used for communication between a patient and medical operating staff during a medical magnetic resonance examination. The patient call ball may include an actuation section configured to be actuated (e.g., pressed) by a patient and a signal unit configured to transmit a communication signal when the actuation section is actuated. The patient call ball may include a magnetic field sensor unit that includes a magnetic field sensor and an activation unit. The activation unit may be configured to activate the signal unit in response to a value of a magnetic field measured by the magnetic field sensor being greater than a limit value.

### CROSS REFERENCE TO RELATED APPLICATIONS

This patent application claims priority to European Patent Application No. 24188010.3, filed Jul. 11, 2024, which is incorporated herein by reference in its entirety.

### BACKGROUND

### Field

The present disclosure relates to a patient call ball which is designed for communication between a patient and medical operating staff during a medical imaging examination and which comprises an actuation section which is designed to be actuated, in particular to be pressed, by a patient, and a signal unit, which is designed to transmit a signal when the actuation section is pressed. In addition, the present disclosure relates to a magnetic resonance apparatus having a patient call ball. The disclosure further relates to a method for using a patient call ball for a magnetic resonance examination.

### Related Art

During a magnetic resonance examination, the patient is positioned in a patient receiving zone of a scanner unit of a magnetic resonance apparatus. The scanner unit is arranged inside an examination room, the examination room being shielded externally with respect to RF radiation. On the other hand, a member of the medical operating staff supervising the magnetic resonance examination, for example a physician, is situated during the magnetic resonance examination in a control room which is constructed separately from the examination room. Conventionally, a patient call ball is known for communicating information from the patient to the medical operating staff during the magnetic resonance examination. The patient holds said patient call ball in their hand during the magnetic resonance examination and is able to press it if necessary, the pressing of the patient call ball causing a signal to be sent to the medical operating staff. By pressing the ball, the patient can draw attention to an emergency situation and/or ask the medical operating staff for attention.

Conventional patient call balls are mainly of pneumatic design. In such cases the patient call balls comprise an air hose, the air hose serving as a means of transmitting a pneumatic signal to a sensor on the patient table due to the patient call ball being pressed. The sensor converts the pneumatic signal into an electrical signal, which is then provided for output to the medical operating staff.

An air hose of a patient call ball must be positioned very carefully on the patient table for a magnetic resonance examination in order to prevent the air hose from slipping down during an entry and/or exit operation of the patient table. An air hose dangling down during a driven movement of the patient table can become jammed between the patient table and an enclosure of the patient receiving zone and get damaged in the process.

Wireless and/or cordless patient call balls can be used to ensure safe movement of the patient table in which the air hose is prevented from becoming jammed. Insofar as said call balls comprise a radiofrequency-based method for transmitting signals and/or information, they are faced with the problem that they must be approved as radio equipment or must comply with EMC (electromagnetic compatibility) limit values. Because the patient call ball is no longer connected mechanically to the scanner unit and/or the patient table, the patient call ball can be taken along everywhere, including outside the examination room, and must therefore also comply with required limit values outside the RF-shielded examination room.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 shows a magnetic resonance apparatus with a patient call ball according to one or more exemplary embodiments of the present disclosure.

FIG. 2 shows a section through the patient call ball according to one or more exemplary embodiments of the present disclosure.

FIG. 3 is a flowchart of a method for using a patient call ball for a magnetic resonance examination according to one or more exemplary embodiments of the present disclosure.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

An object of the present disclosure is to reduce or minimize any damage caused by the patient call ball to components and/or devices which may be arranged outside the examination room.

A first aspect of the disclosure relates to a patient call ball which is designed for communication between a patient and medical operating staff during a medical magnetic resonance examination and which may comprise an actuation section, which is designed to be actuated, in particular to be pressed, by a patient, and a signal unit, which is designed to transmit a communication signal when the actuation section is pressed. It is proposed that the patient call ball may comprise a magnetic field sensor unit having a magnetic field sensor and an activation unit, the activation unit being designed to activate the signal unit if a value of a magnetic field measured by the magnetic field sensor is greater than an activation limit value.

The patient call ball may be configured as a cordless and/or wireless patient call ball. A cordless and/or wireless data transmission can take place in this case via a radio link, for example a Bluetooth connection, and/or by transmitting radiofrequency signals and/or by transmitting pilot tone signals and/or by further wireless data transmission methods deemed useful by the person skilled in the art. In an exemplary embodiment, the signal unit has a transmit antenna for transmitting a communication signal. In this case the signal unit sends the communication signal based on electromagnetic waves or electromagnetic coupling.

The patient call ball may comprise an actuation section, the actuation section having at least one pushbutton and/or at least one pressing section, the at least one pushbutton and/or the at least one pressing section being designed to be pressed by a user, such as the patient. In an exemplary embodiment, the patient call ball has a single pushbutton and/or a single pressing section for transmitting a communication signal. The patient call ball may be of a size that allows the patient call ball to be held, such as cupped, in a hand (e.g., a single hand) of the patient. In an exemplary embodiment, it is possible for the patient to grip the patient call ball with one hand, such as to grip at least 50% of the patient call ball and preferably at least 70% of the patient call ball.

The patient call ball may communicate with a communication unit which is arranged inside the control room and is designed to output the communication signal to a user, in particular to a member of the medical operating staff supervising the magnetic resonance examination. The communication signal can notify the user, in particular the member of the medical operating staff, if the patient experiences discomfort and/or an emergency situation occurs during the magnetic resonance examination. When the actuation section of the patient call ball is actuated (e.g., pressed) the medical operating staff receives corresponding output information at a communication element, in particular an output element, of the communication unit.

The magnetic field sensor unit may comprise the magnetic field sensor and the activation unit. The magnetic field sensor may comprise a Hall sensor, for example. Moreover, the magnetic field sensor is designed to detect a magnetic field. In this way it can be determined whether the patient call ball is situated in the vicinity of the magnetic resonance apparatus, in particular of a scanner unit of the magnetic resonance apparatus. Further, the magnetic field sensor unit may comprise a 3D magnetic field sensor, for example a 3D Hall sensor, so that it is also possible to detect a position of the patient call ball.

The activation unit is designed in the first instance for comparing the magnetic field detected by the magnetic field sensor with a limit value, in particular a predefined activation limit value. The activation limit value may be tailored to fit the magnetic resonance apparatus, in particular in line with a magnetic field of the magnetic resonance apparatus. In this case there may also be different activation limit values for the patient call ball available for different magnetic resonance apparatuses having different magnetic field strengths. As soon as the magnetic field sensor detects a magnetic field whose value is greater than the activation limit value predefined by the activation unit, the signal unit is activated by the activation unit. The activation limit value may be designed in such a way that an exceeding of the activation limit value by a measured value of the magnetic field strength is possible only inside the examination room. In an exemplary embodiment, in this case, an exceeding of the activation limit value by a measured value of the magnetic field strength, and consequently an activation of the signal unit, is possible only inside a patient receiving zone of a magnetic resonance apparatus or at a small distance from the scanner unit of the magnetic resonance apparatus, for example a distance of less than 2 m, preferably of less than 1.5 m from the scanner unit.

For this purpose, the activation unit may comprise a control unit and/or a computing unit. The control unit and/or computing unit can in this case have a corresponding circuit and/or also a corresponding computing module. The signal unit may be activated automatically by the activation unit.

The disclosure has the advantage that the signal unit can be activated only inside the magnetic field of the magnetic resonance apparatus and therefore close to the scanner unit of the magnetic resonance apparatus. In this way it is also ensured that the patient call ball can only be activated inside the examination room. An unintentional actuation, in particular pressing, of the actuation section when the patient call ball is disposed outside of the examination room therefore results in no communication signal being transmitted since in such a case the signal unit is not activated and also cannot be activated. Interference affecting other devices that would be caused by transmitting a communication signal outside of the examination room can advantageously be prevented as a result. A further advantage is that the examination room is shielded externally with respect to radiofrequency radiation such that an activation of the signal unit and a transmission of communication signals are possible only within a shielded environment. A communication signal transmitted by the patient call ball can be attenuated by at least 10 dB to 40 dB by means of a shielded cabin that is arranged around the examination room. In this way, a high safety standard can be provided for a magnetic resonance apparatus having a wireless and/or cordless patient call ball.

In an advantageous development of the patient call ball according to the disclosure, it is proposed that the signal unit is designed for transmitting a registration signal. In an exemplary embodiment, the signal unit is designed for transmitting the registration signal at the start of an activation by the activation unit. The registration signal may be different from the communication signal such that the signal unit is designed for transmitting two different signals, namely the communication signal and a registration signal different therefrom. The registration signal can in this case be captured by a communication unit which is available to the medical operating staff for communication with the patient during the magnetic resonance examination. In an exemplary embodiment, registration information based on the registration signal is output to the user, in particular a member of the medical operating staff, by the communication unit. In this way, the user, in particular a member of the medical operating staff, can be informed about a successful registration and activation of the patient call ball for a magnetic resonance examination. In particular, the registration signal, and thus the registration information output to the user, in particular to the member of the medical operating staff, indicates that the patient call ball is ready for use.

In an advantageous development of the patient call ball according to the disclosure, it is proposed that the patient call ball may comprise an output unit which is designed for outputting registration information. The registration information may be generated by the activation unit and provided to the output unit for output. In this case the output unit may comprise an optical output unit for optical output of the registration information or an acoustic output unit for acoustic output of the registration information. For example, the optical output unit may indicate a successful registration and/or activation of the patient call ball to the user and/or the patient by means of a simple change of color of a light indicator. If the output unit may comprise an acoustic output unit, a successful registration and/or activation of the patient call ball can be indicated to the user and/or the patient by transmission of a signal tone, for example.

In an advantageous development of the patient call ball according to the disclosure, it is proposed that the signal unit may comprise a receive unit for receiving an acknowledgement signal. In an exemplary embodiment, the acknowledgement signal is sent by the communication unit, which is available to the medical operating staff during a magnetic resonance examination for communication with the patient, to the patient call ball, in particular to the signal unit of the patient call ball, following receipt of the registration signal. The received acknowledgement signal can additionally be output directly via the output unit of the patient call ball so that a user, for example a member of the medical operating staff, receives a notification already during the preparation of the patient for a magnetic resonance examination that the patient call ball has been successfully activated and registered. This can also simplify a workflow for the medical operating staff since there is no need for the medical operating staff to leave the examination room in order to check for a successful activation and registration of the patient call ball.

In an advantageous development of the patient call ball according to the disclosure, it is proposed that the activation unit is designed for deactivating the signal unit as soon as a value of a magnetic field detected by the magnetic field sensor is less than a deactivation limit value. The activation limit value on which an activation of the signal unit by the activation unit is based can in this case be the same as the deactivation limit value on which a deactivation of the signal unit by the activation unit is based. Furthermore, the two limit values may also be defined as different. Further, the activation limit value for an activation of the signal unit may be greater than the deactivation limit value for a deactivation of the signal unit. The signal unit may be deactivated automatically by the activation unit. Following a deactivation, the signal unit is in an inactive mode or safety mode such that a transmission of a signal is not possible or is prevented if the actuation section is actuated, in particular pressed. By this means, damage to other equipment due to the patient call ball, for example by pressing the actuation section in an inactive mode or the safety mode of the signal unit, can advantageously be prevented. In particular, a transmission of a signal by the signal unit outside of an area having a high magnetic field and/or outside of the examination region can advantageously be prevented in this case.

A further aspect of the disclosure relates to a magnetic resonance apparatus comprising a first communication unit, which is arranged inside a control room, and a second communication unit, which is arranged inside an examination room, wherein the second communication unit has a patient call ball and wherein the first communication unit and the patient call ball are designed for exchanging communication signals.

The magnetic resonance apparatus may comprise a medical and/or diagnostic magnetic resonance apparatus which is configured and/or designed for acquiring medical and/or diagnostic image data, in particular medical and/or diagnostic magnetic resonance image data, of a patient. For this purpose, the magnetic resonance apparatus may comprise a scanner unit, which is arranged inside the examination room. The scanner unit may comprise a magnet unit for acquiring the medical and/or diagnostic image data. In this arrangement, the scanner unit, in particular the magnet unit, advantageously may comprise a main magnet, a gradient coil unit and a radiofrequency antenna unit. The radiofrequency antenna unit is permanently arranged inside the scanner unit and is configured and/or designed for transmitting an excitation pulse.

The main magnet is designed for generating a homogeneous main magnetic field having a defined magnetic field strength, such as, for example, having a magnetic field strength of 3 T or 1.5 T, etc. In particular, the main magnet is designed to generate a strong and constant main magnetic field. The homogeneous main magnetic field may be arranged and/or to be found inside a patient receiving zone of the magnetic resonance apparatus. The gradient coil unit is designed for generating magnetic field gradients which are used for spatial encoding during an imaging procedure.

The patient receiving zone of the magnetic resonance apparatus is configured and/or designed to accommodate the patient, in particular the region of the patient that is to be examined, for a medical magnetic resonance examination. The patient receiving zone may comprise the area available to the patient during a magnetic resonance examination. For this purpose, the patient receiving zone is designed for example in the shape of a cylinder and/or is cylindrically enclosed by the scanner unit, in particular the magnet unit, of the magnetic resonance apparatus.

A field of view (FoV) and an isocenter of the magnetic resonance apparatus may be arranged inside the patient receiving zone. The FoV may comprise an acquisition range of the magnetic resonance apparatus within which the conditions for an acquisition of medical image data, in particular of magnetic resonance image data, are present inside the patient receiving zone, such as a homogeneous main magnetic field, for example. The isocenter of the magnetic resonance apparatus may comprise the area and/or point within the magnetic resonance apparatus which have/has the optimal and/or ideal conditions for the acquisition of medical image data. In particular, the isocenter may comprise the most homogeneous magnetic field area within the magnetic resonance apparatus.

For the purpose of positioning the patient, in particular the region of the patient that is to be examined, inside the patient receiving zone, the magnetic resonance apparatus may comprise the patient support and positioning device. The patient support and positioning device is designed to position and support the patient inside the patient receiving zone. For this purpose, the patient support and positioning device may comprise a movable patient table which is designed to be movable in particular inside the patient receiving zone of the magnetic resonance apparatus. In an exemplary embodiment, the patient table is in this case designed to be movable in the longitudinal direction of the patient receiving zone and/or in the z-direction inside the patient receiving zone. For a magnetic resonance examination, the patient is positioned on the patient table of the patient support and positioning device and ancillary units required for the magnetic resonance examination, in particular the patient call ball, are likewise positioned on the patient table or on the patient and driven into the patient receiving zone.

The medical operating staff is present in the control room during the magnetic resonance examination. The control room is equipped with a user interface and the first communication unit for the purpose of controlling and/or monitoring magnetic resonance examinations. In addition, a host computer of the magnetic resonance apparatus may also be arranged inside the control room. The control room is designed separately from the examination room in which the scanner unit of the magnetic resonance apparatus is arranged. In particular, the examination room is shielded off from the control room with respect to RF radiation.

The first communication unit may comprise an output unit which is designed for outputting a communication signal. The output unit may be embodied as an optical output unit featuring an indicator and/or a display, etc. Additionally, or alternatively, the output unit may also comprise an acoustic output unit. In addition to an output unit, the first communication unit may also comprise an input unit which is designed to input information to the patient. The second communication unit may comprise the patient call ball. In addition to the patient call ball, the second communication unit may also comprise further communication elements, for example optical and/or acoustic output elements, such as in particular headphones and/or a display.

A magnetic resonance apparatus of said type has the advantage that the signal unit can be activated only within the magnetic field of the magnetic resonance apparatus and therefore close to the scanner unit of the magnetic resonance apparatus. In this way it is also ensured that the patient call ball can only be activated within the examination room. An unintentional actuation, in particular pressing, of the actuation section when the patient call ball is arranged outside of the examination room does not therefore lead to a transmission of a communication signal since in such a case the signal unit is not activated and also cannot be activated. Interference affecting other devices that would be caused by transmitting a communication signal outside of the examination room can advantageously be prevented as a result. A further advantage is that the examination room is shielded off externally with respect to radiofrequency radiation such that an activation of the signal unit and a transmission of communication signals are possible only within a shielded environment. A communication signal transmitted by the patient call ball can be attenuated by at least 10 dB to 40 dB by means of a shielded cabin that is arranged around the examination room. In this way, a high safety standard can be provided for a magnetic resonance apparatus having a wireless and/or cordless patient call ball.

The advantages of the magnetic resonance apparatus according to the disclosure substantially correspond to the advantages of the patient call ball according to the disclosure, which are described in detail hereinabove. Features, advantages or alternative embodiments mentioned in this context can equally be applied to the other claimed subject matters and vice versa.

In an advantageous development of the magnetic resonance apparatus according to the disclosure, it is proposed that the first communication unit may comprise a receive unit, which is designed to receive signals of the patient call ball, and a transmit unit, which is designed to send signals to the patient call ball. This enables a simple exchange of signals between the first communication unit and the patient call ball. In particular, in addition to communication signals and/or registration signals being received by means of the first communication unit, an acknowledgement signal for confirming an activation and/or registration can also be sent to the patient call ball in this way.

A further aspect of the disclosure relates to a method for using a patient call ball for a magnetic resonance examination comprising a patient call ball and a magnetic resonance apparatus, wherein the method may comprise the following method steps:
measuring a value of a magnetic field at the patient call ball by means of a magnetic field sensor of the patient call ball, and
activating a signal unit of the patient call ball by means of an activation unit of the patient call ball as soon as the measured value of the magnetic field exceeds an activation limit value.

In order for the signal unit to be activated by the activation unit, the patient call ball may be introduced into the magnetic field of the magnetic resonance apparatus. The patient call ball may be introduced into the magnetic field of the magnetic resonance apparatus during a preparation of a patient for a magnetic resonance examination. In the process, the patient call ball is handed to the patient for signaling emergency situations during the magnetic resonance examination. Subsequently, the patient is moved forward together with the patient call ball into the patient receiving zone by means of the patient table.

This ensures that the signal unit can be activated only inside the magnetic field of the magnetic resonance apparatus and therefore close to the scanner unit of the magnetic resonance apparatus. In this way it is also ensured that the patient call ball can only be activated inside the examination room. An unintentional actuation, in particular pressing, of the actuation section when the patient call ball is disposed outside of the examination room therefore leads to no communication signal being transmitted since in such a case the signal unit is not activated and also cannot be activated. Interference affecting other devices that would be caused by transmitting a communication signal outside of the examination room can advantageously be prevented as a result. A further advantage is that the examination room is shielded off externally with respect to radiofrequency radiation such that an activation of the signal unit and a transmission of communication signals are possible only within a shielded environment. A communication signal transmitted by the patient call ball can be attenuated by at least 10 dB to 40 dB by means of a shielded cabin that is arranged around the examination room. In this way, a high safety standard can be provided for a magnetic resonance apparatus having a wireless and/or cordless patient call ball.

The advantages of the method according to the disclosure for using a patient call ball for a magnetic resonance examination substantially correspond to the advantages of the patient call ball according to the disclosure and the magnetic resonance apparatus according to the disclosure, which are described in detail hereinabove. Features, advantages or alternative embodiments mentioned in this context can also be applied to the other claimed subject matters and vice versa.

In an advantageous development of the method according to the disclosure, it is proposed that the activation of the signal unit may comprise a transmission of a registration signal by the signal unit, the registration signal being detected by a communication unit of the magnetic resonance apparatus. In this case the registration signal is transmitted at the start of the activation of the signal unit. The registration signal is designed differently here from the communication signal such that the signal unit is embodied for transmitting two different signals, namely the communication signal and a registration signal different therefrom. Information relating to an activation of the patient call ball on the magnetic resonance apparatus can be present as a result of the registration signal. This also enables a user, in particular a member of the medical operating staff, to check whether the patient call ball is ready for use for the upcoming magnetic resonance examination.

In an advantageous development of the method according to the disclosure, it is proposed that call ball information is transmitted to the communication unit of the magnetic resonance apparatus in addition to the registration signal. In an exemplary embodiment, the call ball information is transmitted to the communication unit of the magnetic resonance apparatus by the signal unit of the patient call ball. The call ball information may comprise a type number and/or serial number of the patient call ball. This embodiment enables the patient call ball to be checked to determine whether it is approved for the magnetic resonance apparatus or not. Particularly in institutions, for example hospitals, having multiple magnetic resonance apparatuses, multiple patient call balls are also available in most cases, so checking a compatibility between magnetic resonance apparatus and patient call ball can be of advantage. Furthermore, updates, for example software updates, for the patient call ball can also be provided on the basis of the provided call ball information.

In an advantageous development of the method according to the disclosure, it is proposed that the registration information and/or the call ball information is output on the communication unit of the magnetic resonance apparatus. For this purpose, the communication unit may comprise an output unit, for example an optical output unit, such as a display and/or an indicator element. For example, the registration information can be visualized by overlaying and/or displaying a call ball icon and/or by changing a displayed color of a displayed and/or superimposed call ball icon and/or changing a representation of a displayed and/or superimposed call ball icon. Alternatively hereto, the output unit may also comprise an acoustic output unit, for example a loudspeaker. By output of the call ball information, in particular an optical output of the call ball information, it is also possible for the user, in particular a member of the medical operating staff, to check whether the patient call ball used for the upcoming magnetic resonance examination is compatible with the selected magnetic resonance apparatus or not.

In an advantageous development of the method according to the disclosure, it is proposed that output information is output on the patient call ball, the output of the output information being triggered by an activation of the signal unit and/or a transmission of a registration signal by the signal unit. The patient call ball may comprise an output unit for outputting the output information. The output unit may comprise an optical output unit, for example a LED display. The display and/or output of the output information can be realized for example by changing a color of the output unit or lighting up the output unit. Outputting the output information directly on the patient call ball enables the user, in particular a member of the medical operating staff, to be notified about the activation and/or registration already during the preparation of the patient for the upcoming magnetic resonance examination. In particular, the medical operating staff can be informed directly about the activation and/or registration when the patient call ball is introduced into the magnetic field. A workflow can also be improved by this means since the medical operating staff is alerted immediately about a failed activation and/or registration, for example, and not just at the communication unit of the magnetic resonance apparatus inside the control room.

In an advantageous development of the method according to the disclosure, it is proposed that the activation of the signal unit may comprise a transmission of an acknowledgement signal by a communication unit of the magnetic resonance apparatus, the acknowledgement signal being detected by the signal unit and output on the patient call ball. The acknowledgement signal may be captured by means of the signal unit, which may comprise a receive unit for this purpose. The acknowledgement signal may be transmitted by the communication unit which is arranged inside the control room and which is available to the medical operating staff for communication with the patient. The received acknowledgement signal may be output by means of the output unit, in particular the optical output unit, of the patient call ball. The acknowledgement signal can be displayed and/or output for example by changing a color of the output unit or lighting up the output unit. The acknowledgement signal may be used to inform the user, in particular a member of the medical operating staff, about a successful activation and/or registration of the patient call ball. The output of the acknowledgement information directly on the patient call ball enables the user, in particular a member of the medical operating staff, to be notified about the activation and/or registration already during the preparation of the patient for the upcoming magnetic resonance examination. In particular, the medical operating staff can be informed directly about the activation and/or registration when the patient call ball is introduced into the magnetic field. A workflow can also be improved by this means since the medical operating staff is alerted immediately about a failed activation and/or registration, for example, and not just at the communication unit of the magnetic resonance apparatus inside the control room.

In an advantageous development of the method according to the disclosure, it is proposed that the signal unit is deactivated by means of the activation unit as soon as a value of the detected magnetic field is less than a deactivation limit value. In this way, a high safety standard for the patient call ball can be provided since the latter is deactivated as soon as it is removed from the magnetic field and consequently can also no longer transmit a signal. The deactivation limit value for a deactivation of the signal unit can in this case be the same as the activation limit value that is used for an activation of the signal unit. Furthermore, the deactivation limit value for a deactivation of the signal unit may also be different from the activation limit value for an activation of the signal unit.

In an advantageous development of the method according to the disclosure, it is proposed that the activation unit is designed in such a way that an activation of the signal unit and/or a deactivation of the signal unit may comprise a delay effect. By means of the delay effect it is possible to prevent the patient call ball from corresponding to a position in the magnetic field of the magnetic resonance apparatus, wherein the position corresponds to a value of the magnetic field that lies in the range of the activation limit value and/or of the deactivation limit value, the signal unit being activated or deactivated each time there are slight changes in the position of the patient call ball. This can also contribute toward a resource-saving use of the patient call ball since the signal unit in particular is not subject to a constant activation and deactivation. For example, a time delay can be superimposed on an activation process and/or a deactivation process so that the activation and/or deactivation of the signal unit take/takes place sometime after the limit value is exceeded and/or undershot. In this case the time delay can comprise a few seconds. Furthermore, the delay can also be affected by the two limit values being different. If, for example, the activation limit value is greater than the deactivation limit value, an active signal unit is not immediately deactivated as soon as the measured value of the magnetic field falls below the activation limit value. Further, an inactive signal unit is also not immediately activated as soon as the measured value of the magnetic field exceeds the deactivation limit value.

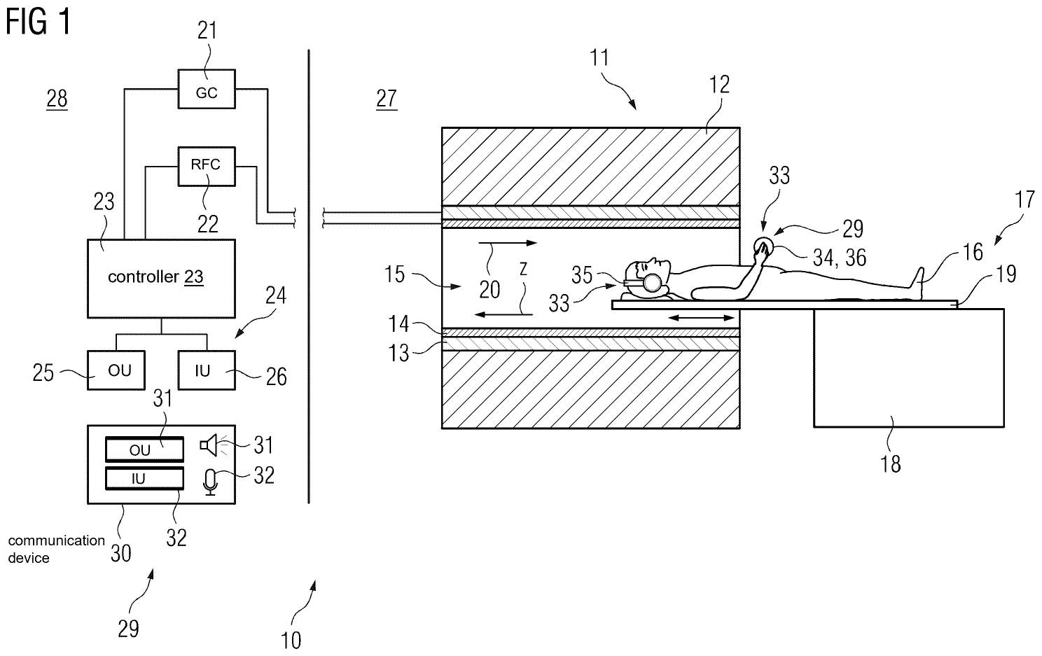

FIG. 1 shows a magnetic resonance apparatus 10 in a schematic view. The magnetic resonance apparatus 10 may comprise a scanner unit designed as a magnet unit 11 having a main magnet 12, a gradient coil unit 13 and a radiofrequency antenna unit 14. In addition, the magnetic resonance apparatus 10 has a patient receiving zone 15 for accommodating a patient 16 for a magnetic resonance examination. In the present exemplary embodiment, the patient receiving zone 15 is designed in the shape of a cylinder and is cylindrically enclosed by the magnet unit 11 in a circumferential direction. In principle, however, an embodiment of the patient receiving zone 15 differing therefrom is conceivable at any time.

The magnetic resonance apparatus 10 has a patient support and positioning device 17 for positioning the patient 16, in particular a region of the patient 16 that is to be examined, inside the patient receiving zone 15. The patient support and positioning device 17 has a base unit 18 and a patient table 19 that is movable relative to the base unit 18. The patient table 19 is designed for positioning the patient 16, in particular the region of the patient 16 that is to be examined, movably inside the patient receiving zone 15. In particular, the patient table 19 is in this case mounted so as to be movable in the direction of a longitudinal dimension of the patient receiving zone 15 and/or in the z-direction.

The main magnet 12 of the magnet unit 11 is designed for generating a strong and in particular constant main magnetic field 20. In this case the main magnet 12 can be embodied for example as a superconducting main magnet 12 or also as a permanent magnet. The gradient coil unit 13 of the magnet unit 11 is designed for generating magnetic field gradients which are used for spatial encoding during an imaging procedure. The gradient coil unit 13 is controlled by means of a gradient control unit 21 of the magnetic resonance apparatus 10. The radiofrequency antenna unit 14 of the magnet unit 11 is designed to excite a polarization which establishes itself in the main magnetic field 20 generated by the main magnet 12. The radiofrequency antenna unit 14 is controlled by a radiofrequency antenna control unit 22 of the magnetic resonance apparatus 10 and radiates radiofrequency magnetic resonance sequences into the patient receiving zone 15 of the magnetic resonance apparatus 10.

The magnetic resonance apparatus 10 may comprise a system control unit 23 for controlling the main magnet 12, the gradient control unit 21 and the radiofrequency antenna control unit 22. The system control unit 23 centrally controls the magnetic resonance apparatus 10, such as for performing a predetermined imaging gradient echo sequence, for example. In addition, the system control unit 23 may comprise an evaluation unit (not shown in more detail) for evaluating medical image data acquired during the magnetic resonance examination.

The magnetic resonance apparatus 10 further may comprise a user interface 24 which is connected to the system control unit 23. Control information such as imaging parameters, for example, as well as reconstructed magnetic resonance images can be displayed on a output unit (visualization unit) 25, for example on at least one monitor, of the user interface 24 for medical operating staff. The user interface 24 also has an input unit 26 by means of which information and/or parameters can be input by a member of the medical operating staff during a measurement procedure.

The scanner unit 11 of the magnetic resonance apparatus 10 is arranged together with the patient support and positioning device 17 inside an examination room 27. The system control unit 22, on the other hand, is arranged together with the user interface 23 inside a control room 28. The control room 28 is implemented separately from the examination room 27. In particular, the examination room 27 is shielded off from the control room 28 with respect to radiofrequency radiation. During a magnetic resonance examination, the patient 13 is located inside the examination room 27, whereas the medical operating staff is located inside the control room 28.

For communication between the patient 16 and a member of the medical operating staff, for example a physician, during a magnetic resonance examination, the magnetic resonance apparatus 10 may comprise a communication system 29. The communication system 29 may comprise a first communication unit 30, which is arranged inside the control room 28 and is available to the medical operating staff for communication with the patient 16 during the magnetic resonance examination. In the present exemplary embodiment, the first communication unit 30 has an output unit 31 and an input unit 32. Information from the patient 16 to the medical operating staff can be output by means of the output unit 31. In this arrangement, the output unit 31 can comprise at least one acoustic output element, such as a loudspeaker, for example, and/or at least one visual output element, such as a display or a monitor, for example. The input unit 32 can in this case comprise an input element, such as, for example, a microphone and/or a keyboard, etc.

The communication system 29 additionally may comprise a second communication unit 33, which is arranged inside the examination room 27. The second communication unit 33 is available to a patient 16 for communication with the medical operating staff during a magnetic resonance examination. The second communication unit 33 may comprise an input unit 34 and an output unit 35. The output unit 35 may comprise an acoustic output unit, such as headphones, for example. In addition, the output unit 35 may also comprise a visual output element, such as a display, for example, which is arranged inside the patient receiving zone 15. The input unit 34 has an input element which is embodied as a patient call ball 36.

The first communication unit 30 and the second communication unit 33 are designed to exchange communication signals during a communication of the patient 16 with the medical operating staff. In an exemplary embodiment, the communication between the patient call ball 36 and the first communication unit 30 is affected in this case by wireless and/or cordless means. For example, a cordless and/or wireless data transmission can be implemented via a radio link, for example a Bluetooth connection, and/or by transmitting radiofrequency signals and/or by transmitting pilot tone signals and/or by further cordless data transmission methods deemed useful by the person skilled in the art. For this purpose, the first communication unit 30 has a receive unit which is designed to receive signals from the patient call ball 36. In addition, the first communication unit 30 may also comprise a transmit unit for transmitting a signal to the patient call ball 36.

The illustrated magnetic resonance apparatus 10 may of course comprise further components which are typically included in magnetic resonance apparatuses 10. Furthermore, a general mode of operation of a magnetic resonance apparatus 10 is known to the person skilled in the art so a detailed description of the further components is dispensed with.

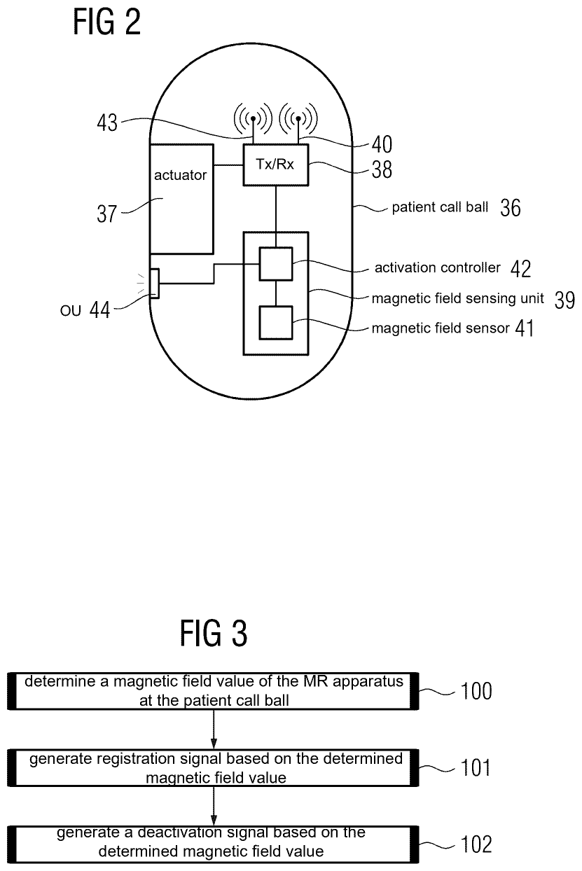

FIG. 2 shows the patient call ball 36 in more detail in a sectional view. The patient call ball 36 may have a shape and a size that allow it to be held and operated and/or actuated by a hand of the patient 16. In an exemplary embodiment, it is possible for the patient 16 to grip the patient call ball 36 with one hand, in particular to grip at least 50% of a surface of the patient call ball 36 and preferably at least 70% of a surface of the patient call ball 36. The patient call ball 36 may comprise an actuation section (actuator) 37, a signal unit (transceiver) 38 and a magnetic field sensor unit 39. The actuation section 37 may be configured to be actuated, such as be pressed, by means of one hand of the patient 16. In this case, the actuation section 37 may comprise a pushbutton and/or a pressing section. In an exemplary embodiment, the patient call ball 36 comprises processing circuitry that is configured to perform one or more functions of the patient call ball 36. Additionally, or alternatively, one or more components of the patient call ball 36 may comprise processing circuitry that is configured to perform one or more respective functions of the component(s).

The signal unit (transceiver) 38 may be configured to transmit and/or receive signals. In particular, the signal unit 38 may be configured to transmit a communication signal when the actuation section 37, in particular the pushbutton and/or the pressing section, is pressed. At the same time, the signal unit 38 may be configured for cordless and/or wireless transmission of signals and may comprise a transmit unit (transmitter) 40 and/or a transmit antenna for this purpose.

The magnetic field sensor unit 39 may comprise a magnetic field sensor 41 and an activation unit (activation controller) 42. The magnetic field sensor 41 may be configured to measure a magnetic field value at the location and/or position at which the patient call ball 36 is located. In this case, the magnetic field sensor 41 may comprise a Hall sensor for detecting a magnetic field. In this case, the Hall sensor may also comprise a 3D Hall sensor. The activation unit 42 may be configured to activate or otherwise control the signal unit 38 when the patient call ball 36 is introduced into a magnetic field of a magnetic resonance apparatus 10, an activation being performed based on the measured magnetic field value. For example, the activation may be in response to a value of the magnetic field measured by the magnetic field sensor 41 being greater than an activation limit value.

Furthermore, the activation unit 42 may be configured to deactivate the patient call ball 36 when the patient call ball 36 is removed from a magnetic field of the magnetic resonance apparatus 10, a deactivation being performed based on the measured magnetic field value. For example, the deactivation may be in response to a value of the magnetic field measured by the magnetic field sensor 41 being less than a deactivation limit value. The deactivation limit value for deactivating the signal unit 38 may at the same time comprise a value that is the same as a value of the activation limit value for the activation of the signal unit 38. Moreover, the deactivation limit value and the activation limit value may also comprise an identical value differently. An activation of the signal unit 38 by the activation unit 42 may, in this case, also comprise a transmission of a registration signal by the signal unit 38 to the first communication unit 30. Furthermore, a deactivation of the signal unit 38 by the activation unit 42 may, in this case, also comprise a transmission of a logoff signal by the signal unit 38 to the first communication unit 30.

The activation unit 42 may comprise a controller and/or processing circuitry configured to monitor the magnetic field and to control the signal unit 38. In this regard, the controller and/or processing circuitry may comprise a corresponding circuit and/or a corresponding computing module which are/is configured to activate and/or deactivate the signal unit 38. In an exemplary embodiment, the signal unit 38 is activated and deactivated automatically by the activation unit 42.

In the present exemplary embodiment, the signal unit 38 may also comprise a receiver 43 and/or a receive antenna which are/is configured to receive one more signals, such as an acknowledgement signal transmitted by the first communication unit 30.

In an exemplary embodiment, the patient call ball 36 may comprise an output unit (output interface) 44. The output unit 44 may comprise an optical output unit for optical and/or visual output of registration information. In an exemplary embodiment, the optical output unit may comprise an LED display which, by being lit up and/or by a change of color in a light display, displays the registration information, in particular a successful registration and/or activation, of the patient call ball 36 to the medical operating staff and/or the patient 16. In an exemplary embodiment, the output unit 44 may additionally or alternatively comprise an acoustic output unit (e.g., speaker) and/or a haptic output unit (e.g., haptic engine). In an exemplary embodiment, the patient call ball 36 may omit the output unit 44.

The patient call ball 36 may comprise further components which are typically included in patient call balls 36. Furthermore, the general mode of operation of a patient call ball 36 is known to the person skilled in the art so a detailed description of the further components is omitted for brevity.

FIG. 3 schematically shows a method for using a patient call ball 36 for a magnetic resonance examination. In this case, initially, in a first method step 100, a value of the magnetic field of the magnetic resonance apparatus 10 at the patient call ball 36 is measured by means of the magnetic field sensor 41 of the patient call ball 36 and forwarded to the activation unit 42. A value of the magnetic field at the location of the patient call ball 36 is constantly measured by means of the magnetic field sensor 41 and forwarded to the activation unit 42.

In a second method step 101, the signal unit 38 of the patient call ball 36 is activated by means of the activation unit 42 as soon as the value of the measured magnetic field exceeds an activation limit value. In this case the measured values of the magnetic field are constantly compared with the activation limit value by the activation unit 42. The activation limit value may in this case be defined in such a way that the measured value of the magnetic field exceeds the activation limit value only when the patient table 19 is driven together with the patient call ball 36 into the patient receiving zone 15 such that no activation of the signal unit 38 can take place outside of the patient receiving zone 15 or at a greater distance from the scanner unit, for example a distance from the scanner unit of more than 2 m.

In this second method step 101, the activation of the signal unit 38 by the activation unit 42 additionally may comprise a transmission of a registration signal by the signal unit 38. In this case, as soon as the measured value of the magnetic field exceeds the activation limit value, a registration signal is transmitted by the signal unit 38. In this case the generation and/or transmission of the registration signal are/is controlled by means of the activation unit 42. The registration signal is captured by the first communication unit 30 and in this way it is signaled to the first communication unit 30 that the patient call ball 36 is now in an active operating mode.

In an alternative embodiment of the patient call ball 36 and/or of the method for using the patient call ball 36 for a magnetic resonance examination, it may also be provided that no registration signal is sent to the first communication unit 30 by the signal unit 38.

In the present exemplary embodiment, call ball information is transmitted to the first communication unit 30 of the magnetic resonance apparatus 10 in addition to the registration signal. In this case the call ball information is also sent to the first communication unit 30 by the signal unit 38. The call ball information may comprise a type number and/or serial number of the patient call ball 36. Furthermore, the call ball information may comprise further information relating to the patient call ball 36. On the basis of the call ball information, the first communication unit 30 can check whether the patient call ball 36 used is compatible with the magnetic resonance apparatus 10 or is approved for use with the magnetic resonance apparatus 10.

In an alternative embodiment of the patient call ball 36 and/or of the method for using the patient call ball 36 for a magnetic resonance examination, it may also be provided that no call ball information is sent to the first communication unit 30 by the signal unit 38.

The activation of the patient call ball 36, in particular the second method step 101, additionally may comprise an output of registration information and/or the call ball information at the first communication unit 30, in particular by means of the output unit 31 of the first communication unit 30. The registration information may comprise an output which signals a successful registration and/or activation of the patient call ball 36 at the first communication unit 30 for the medical operating staff. For example, an icon symbolizing a patient call ball can be superimposed on a display and/or monitor of the first communication unit 30. In addition, an already displayed icon of a patient call ball can be represented and/or displayed in a different color on a display and/or monitor of the first communication unit 30.

In the present exemplary embodiment, the activation of the patient call ball 36, in particular the second method step 101, additionally may comprise a transmission of an acknowledgement signal to the patient call ball 36 by the first communication unit 30. The acknowledgement signal is captured by the signal unit 38, in particular the receive unit 43 of the signal unit 38, of the patient call ball 36. This acknowledgement signal signals to the activation unit 42 that an activation and/or registration of the patient call ball 36 have/has taken place at the first communication unit 30.

In an alternative embodiment of the patient call ball 36 and/or of the method for using the patient call ball 36 for a magnetic resonance examination, it may also be provided that no acknowledgement signal is sent by the first communication unit 30.

In the present exemplary embodiment, the activation of the patient call ball 36, in particular the second method step 101, additionally may comprise that output information is output on the patient call ball 36, in particular on the output unit 44 of the patient call ball 36. In this case the output information informs a user, in particular a member of the medical operating staff and/or the patient, about a successful activation and/or registration of the patient call ball 36 at the first communication unit 30. The output of the output information is in this case triggered by the activation of the signal unit 38 or by a transmission of a registration signal by the signal unit 36 or by a receipt of the acknowledgement signal.

In a further, third method step 102, the activation unit 42 is designed to deactivate the signal unit 38 as soon as a value of the measured magnetic field at the location of the patient call ball 36 is less than a deactivation limit value. This is in particular the case when the magnetic resonance examination has been terminated and the patient 16 together with the patient call ball 36 exits the patient receiving zone 15 of the scanner unit. In this method step 102, the signal unit 38 may also be designed for transmitting a deactivation signal to the first communication unit 30. A deactivation of the signal unit 38 may in this case also be displayed on the output unit 31 of the first communication unit 30. For example, an already displayed icon symbolizing a patient call ball can be hidden on a display and/or monitor. Furthermore, an already displayed icon symbolizing a patient call ball can be represented and/or displayed in a different color on a display and/or monitor of the first communication unit 30.

In an alternative embodiment of the patient call ball 36 and/or of the method for using the patient call ball 36 for a magnetic resonance examination, it may also be provided that no deactivation signal is transmitted by the signal unit 38.

In the present exemplary embodiment, it is furthermore provided that an activation and/or a deactivation of the signal unit 38 by the activation unit 42 comprise/may comprise a delay effect. For example, a time delay can be superimposed on the activation process and/or the deactivation process so that the activation and/or deactivation of the signal unit 38 takes place sometime after the activation limit value is exceeded and/or the deactivation limit value is undershot. In this case the time delay can comprise a few seconds. Furthermore, the delay can also be affected by the two limit values being different. If, for example, the activation limit value is greater than the deactivation limit value, an active signal unit 38 is not immediately deactivated as soon as the measured value of the magnetic field falls below the activation limit value. Further, an inactive signal unit 38 is also not immediately activated as soon as the measured value of the magnetic field exceeds the deactivation limit value.

Although the disclosure has been illustrated and described in more detail based on exemplary embodiments, the disclosure is not limited by the disclosed examples and other variations can be derived herefrom by the person skilled in the art without leaving the scope of protection of the disclosure.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

The various components described herein may be referred to as “modules,” “units,” or “devices.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such modules, units, or devices, as applicable and relevant, may alternatively be referred to herein as “circuitry,” “controllers,” “processors,” or “processing circuitry,” or alternatively as noted herein.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A patient call ball for communication between a patient and medical operating staff during a medical magnetic resonance examination, the patient call ball comprising:
an actuator configured to be actuated by a patient;
transceiver configured to transmit a communication signal based on an actuation of the actuator; and
a magnetic field sensor unit comprising a magnetic field sensor and an activation controller, wherein the activation controller is configured to activate the transceiver based on a value of a magnetic field measured by the magnetic field sensor being greater than an activation limit value.

2. The patient call ball as claimed in claim 1, wherein the transceiver is configured to transmit at least one registration signal.

3. The patient call ball as claimed in claim 1, further comprising an output interface configured to output registration information.

4. The patient call ball as claimed in claim 1, wherein the transceiver comprises a receiver configured to receive an acknowledgement signal.

5. The patient call ball as claimed in claim 1, wherein the activation controller is further configured to deactivate the signal unit based on the value of the magnetic field measured by the magnetic field sensor being less than a deactivation limit value.

6. A magnetic resonance apparatus comprising:
a first communication unit arranged inside a control room; and
a second communication unit arranged inside an examination room, the second communication unit comprising a patient call ball, wherein the first communication unit and the patient call ball are configured to exchange communication signals, the patient call ball comprising:
an actuator configured to be actuated by a patient;
transceiver configured to transmit a communication signal based on an actuation of the actuator; and
a magnetic field sensor unit comprising a magnetic field sensor and an activation controller, wherein the activation controller is configured to activate the transceiver based on a value of a magnetic field measured by the magnetic field sensor and one or more threshold values.

7. The magnetic resonance apparatus as claimed in claim 6, wherein the first communication unit comprises a receiver configured to receive signals from the patient call ball, and a transmitter configured to transmit signals to the patient call ball.

8. A method for using a patient call ball for a magnetic resonance examination, the method comprising:
measuring a value of a magnetic field at the patient call ball using a magnetic field sensor of the patient call ball; and
activating a transceiver of the patient call ball based on the measured value of the magnetic field exceeding an activation limit value.

9. The method as claimed in claim 8, wherein the activation of the transceiver comprises a transmission of a registration signal by the transceiver, wherein the registration signal is captured by a communication unit of the magnetic resonance apparatus.

10. The method as claimed in claim 9, further comprising transmitting call ball information to the communication unit of the magnetic resonance apparatus.

11. The method as claimed in claim 9, wherein the registration signal and/or the call ball information is output at the communication unit of the magnetic resonance apparatus.

12. The method as claimed in claim 8, further comprising outputting output information on the patient call ball based on an activation of the transceiver and/or a transmission of a registration signal by the transceiver.

13. The method as claimed in claim 8, wherein the activation of the transceiver comprises a transmission of an acknowledgement signal by a communication unit of the magnetic resonance apparatus, the acknowledgement signal being captured by the transceiver and output on the patient call ball.

14. The method as claimed in claim 8, wherein the transceiver is deactivated based on the value of the measured magnetic field being less than a deactivation limit value.

15. The method as claimed in claim 14, wherein the activation of the transceiver and/or the deactivation of the transceiver are configured with a delay effect.

16. One or more non-transitory media storing instructions that, when executed by one or more processors, cause the one or more processors to perform the method of claim 8.
